(19) United States
(12) Patent Application Publication
(10) Pub. No.: US 2018/0242709 A1
(43) Pub. Date: Aug. 30, 2018

(54) Roll-On Deodorant with Decorative Effect

(71) Applicant: Corinna Herzmann, Duisburg (DE)

(72) Inventor: Corinna Herzmann, Duisburg (DE)

(21) Appl. No.: 15/899,373

(22) Filed: Feb. 20, 2018

(30) Foreign Application Priority Data
Feb. 24, 2017 (DE) 102017001789.1

(51) Int. Cl. A45D 33/12; A45D 33/18; A45D 34/04; A45D 40/26; A61K 8/02; A61Q 15/00

(52) U.S. Cl. CPC A45D 33/12; A45D 33/18; A45D 34/041; A45D 2034/007; A61K 8/0245; A61Q 15/00; A45D 40/261

(57) ABSTRACT

An attachment element 2 for a container 10 for applying a substance, in particular liquids, free-flowing formulations, pastes or powders, comprising at least one rotationally symmetric body 4 freely moving in a support for applying the substance. The attachment element 2 is connected with a container 10 in which the substance to be applied is contained and transported, by the body 4 to which the substance adheres during a rotary motion, to the outside to apply it, for example, onto skin. To use it for advertising measures or to increase its attractiveness, the body 4 comprises a sealable cavity 5 and at least partially transparent material, wherein the body 4 is at least partially filled with an at least partially transparent liquid and/or solid particles 6. In case of rotation, the liquid in the body 4 or the solid particles 6 are swirled around; a decorative appearance is created.

### CROSS REFERENCE TO RELATED APPLICATIONS

This application claims the priority of DE 102017001789.1 filed on 2017 Feb. 24; this application is incorporated by reference herein in its entirety.

### BACKGROUND

The invention relates to an attachment element for a container for applying a substance, in particular for liquids, free-flowing formulations, pastes or powders, comprising at least one rotationally symmetric body free moving in a support for applying the substance, in particular onto a skin surface.

Generic attachment elements are offered, for example, as roll-on deodorants (roll-on sticks), which are provided for placing them onto a container, preferably a round container with a viscous liquid. Typically, the attachment element is screwed onto the container so that the container is simultaneously sealed. The attachment element has a rotationally symmetric body which is received in a support in a free moving manner. The support normally consists of two half shells which are pressed after the rotationally symmetric body has been received therein. Thereby, the body is securely retained in the support and is able to freely rotate in any direction, and moreover, a small gap is provided between the support and the body by correspondingly selecting the diameter so that in case of a rotary motion of the body, which is furthermore in contact with the substance, the latter may adhere to the surface and will get outside as a result of the rolling motion of the body.

The support encloses the body by more than 50% so that the latter cannot fall out of the support at the top. Less than 50% of the body's surface project from the support and are thus freely accessible so that the substance taken up by the body and adhering to the body is transported, during a rotary motion, outwards through the gap provided between the body and the support. To this end, the body may be additionally provided with indentations where the substance is deposited from the container during a rotary motion. When the attachment element is not in use, it may be closed with a cap so that the substance does not evaporate or cause impurities in a hand bag etc.

The known attachment elements only differ slightly and only offer the possibility of applying a substance.

From US application 2012/0109041 A1, a roll-on deodorant with a container for applying liquids or free-flowing formulations, pastes or powders is known which comprises, apart from the container, a rotationally symmetric body free moving in a support. The body used for this consists of an at least partially transparent material.

### SUMMARY

The invention relates to an attachment element 2 for a container 10 for applying a substance, in particular liquids, free-flowing formulations, pastes or powders, comprising at least one rotationally symmetric body 4 free moving in a support for applying the substance in particular onto a skin surface. The attachment element 2 is here connected with a container 10 in which the substance to be applied is contained and transported, by the body 4 to which the substance adheres during a rotary motion, to the outside to apply it, for example, onto a skin. To use it for advertising measures or to increase its attractiveness, the body 4 comprises a sealable cavity 5 and consists of at least partially transparent material, wherein the body 4 is at least partially filled with an at least partially transparent liquid and/or solid particles 6. In case of rotation, the liquid in the body 4 or the solid particles 6 are swirled around so that a decorative appearance is created which is excellently suited, for example, as an advertising medium.

### DETAILED DESCRIPTION

It is an object of the present invention to provide an attachment element which imparts an optically attractive impression and is moreover suited as an advertising medium.

To achieve this object, the body comprises, according to the invention, a sealable cavity and consists of an at least partially transparent material, the body being at least partially filled with an at least partially transparent liquid and/or solid particles. Further advantageous embodiments of the invention can be taken from the subclaims.

The principle employed when the rotationally symmetric body is used for transferring the substance located in the container onto a skin surface is well-known. What is new is that an at least partially transparent liquid and/or solid particles are contained in the body, wherein the body itself consists of a transparent material and may also be filled only partially. Such a filling may be employed, for example, as they are employed in snow globes which are offered as decorative objects. Attachment elements as roll-on sticks, however, have a practical function and are used very often. By means of the transparent liquid and/or the solid particles, the effect of a snow globe is imitated, and by the given practical benefit, the finished product is made available to a broad circle of interested people due to the light effects that can be achieved. By the rotation and inclination of the body in use, the liquid inside is agitated, and, for example, the snow-imitating particles or comparable particles are swirled up so that the known snow globe effect is created. The liquid may be a transparent or semi-transparent liquid optionally consisting of a plurality of coloured liquids which are not intermingled or are only intermingled temporarily by the rolling motion, which, however, separate again when at rest. Moreover, solid particles may be contained in the liquid which are swirled around in the rotationally symmetric body and thus create the snow effect or comparable effects. As an alternative, larger figurative representations may be deposited inside the rotationally symmetric body and perform a proper motion due to rotation which is possibly slowed down, depending on the liquid. By the movement continued even after the roll-on stick has been put down, this effect is maintained for a certain time and thus attracts the attention of other persons. Thereby, an interesting optical and decorative impression is conveyed which does not only give an incentive to buy but is also an eye-catcher. Here, it is easily possible for the solid particles themselves to be fluorescent or luminescent so that this effect may be temporarily peceived even when it is getting dark or in dark rooms.

In a preferred embodiment, the attachment element is screwed onto a container and has, to this end, an internal thread which corresponds to the external thread of the container. The attachment element itself usually consists of two main parts, which are a support designed as a receiving shell, and a body free moving in the receiving shell. The receiving shell functions to hold the body within the attachment element, for example to prevent it from falling out. The manufacture is on the one hand effected with respect to the rotationally symmetric body with a corresponding filling, and on the other hand preferably by injection moulding for manufacturing the receiving shells as a support which are connected to each other after the body has been inserted. Here, it is possible to create a permanent connection of the two receiving shells by an interference fit, locking clamps, or else by a weld seam. After the attachment element has been produced, it may be subsequently screwed onto the container and is ready for use. Prior to this, the container is filled with a substance which may be transferred onto a skin surface by means of the attachment element.

The use of the attachment element is not restricted. The attachment element may be employed for applying any viscous liquids, for example as a deodorant, a lip balm or perfume. Furthermore, the use of the attachment element for applying viscous bonding materials is conceivable, for example as a roll-on glue stick which can be found in any children's room.

The receiving shells are preferably designed to be annular and consist of plastics. The shape of the receiving shells has to be adapted to the rotationally symmetric body so that the rotationally symmetric body may freely rotate in the shell.

Depending on the application and as desired, the body may be designed in one piece or two pieces and be filled, with accordingly existing techniques, with a transparent liquid and/or free moving solid particles.

In a first preferred embodiment, the body consists of a ball which is embodied in two pieces and filled with a transparent liquid and particles, optionally free from any air bubbles. For the filling operation, a commercially available technique may be employed depending on the intended purpose, the filling material and the shape of the body.

By the use of the attachment element, for example, as a roll-on applicator of a deodorant, the ball is caused to move, and the contained solid particles are swirled up so that the optical effect of a snow ball is created.

The free moving solid particles may consist of any particles which may be employed for decorative purposes, such as pearls, twinkling and/or coloured stars, flakes or advertising elements which are all swirled around by the rotary motion.

It is conceivable to manufacture the body in the form of a ball, a cylinder or an ellipse. The above-mentioned bodies are designed to be rotationally symmetric at least with respect to one axis and may freely move in a receiving shell with a corresponding shape. In contrast, a ball has any desired number of axes of rotation so that this shape is preferably employed.

To further increase the optical effect of the filled body, the attachment element may comprise, in a further preferred embodiment, a cavity which is subdivided by one or several partitions. Each of these spaces is filled with a transparent liquid and the solid particles, such as twinkling and/or coloured pearls, stars, flakes or the like.

In another embodiment of the invention, the spaces within the ball may be filled with coloured liquids. Thus, the attachment element may be used, for example, as an advertising article or for reproducing a certain brand name or a flag. For decorative or commercial purposes, the cavity may be equipped with a structure which has, for example, an image engraved by a laser, a net structure or a symbol of any desired shape. An engraving with an image, for example a brand name, a figurative representation, a photograph or a text which refer to a certain event etc. is conceivable.

In another preferred embodiment of the attachment element, figurines are contained in the cavity. The figurines may be firmly connected to the wall or be freely moving within the cavity.

To be able to ensure the adhesion of the substance to the surface of the rotationally symmetric body and the transportability, the rotationally symmetric body has, in a preferred embodiment, an at least partially rough surface, or indentations in which the substance is temporarily received are provided.

Furthermore, an engraving of structures on the surface of the rotationally symmetric body is conceivable. The structures are to improve the transportability of the substance, wherein said substance may be provided on the outer side or on the inner side. Here, the structure of the surface of the rotationally symmetric body may reflect, alone or in combination with the structure on the inner surface of the rotationally symmetric body, a pattern, an image or, for example, a brand name. It is furthermore conceivable that the structure on the surface of the rotationally symmetric body is combined with the solid particles and/or figurines located in the cavity. Moreover, the surface may be at least partially coloured.

In another preferred embodiment of the attachment element, the structuring on the surface of the rotationally symmetric body may consist of engraved motives or geometric shapes by indentations and elevations.

The particular advantage of the present invention resides in that a conventional roll-on stick is provided, instead of with a usual attachment element, with a novel attachment element whereby a number of effects may be achieved. The roll-on stick here consists of a container in which the substance to be applied is contained which may be transferred to the skin via the rotationally symmetric body. To this end, the container has, for example, a thread so that the attachment element may be screwed on with an internal thread. The attachment element furthermore consists of at least two receiving shells which are permanently connected to each other so that the enclosed body may rotate between the receiving shells while it is otherwise held stationarily. The body itself is preferably made of a transparent material and may be filled. The filling may here be effected with coloured jelly-like liquids, but it is also possible to receive solid particles in the body which are swirled around as a result of the rotation of the body and create a snow globe effect. The colour or solid particles used may comprise a certain imagery and thus lead to an interesting viewing object which may also be employed, for example, as an advertising medium.

### BRIEF DESCRIPTION OF THE DRAWINGS

The invention will be further illustrated again below with reference to the figures.

In the drawings:

FIG. 1 shows a side view of a roll-on stick with the attachment element,

FIG. 2 shows a plan view of the attachment element, and

FIG. 3 shows a side view of a spherical-symmetrical body.

### DETAILED DESCRIPTION OF THE PREFERRED EMBODIMENTS

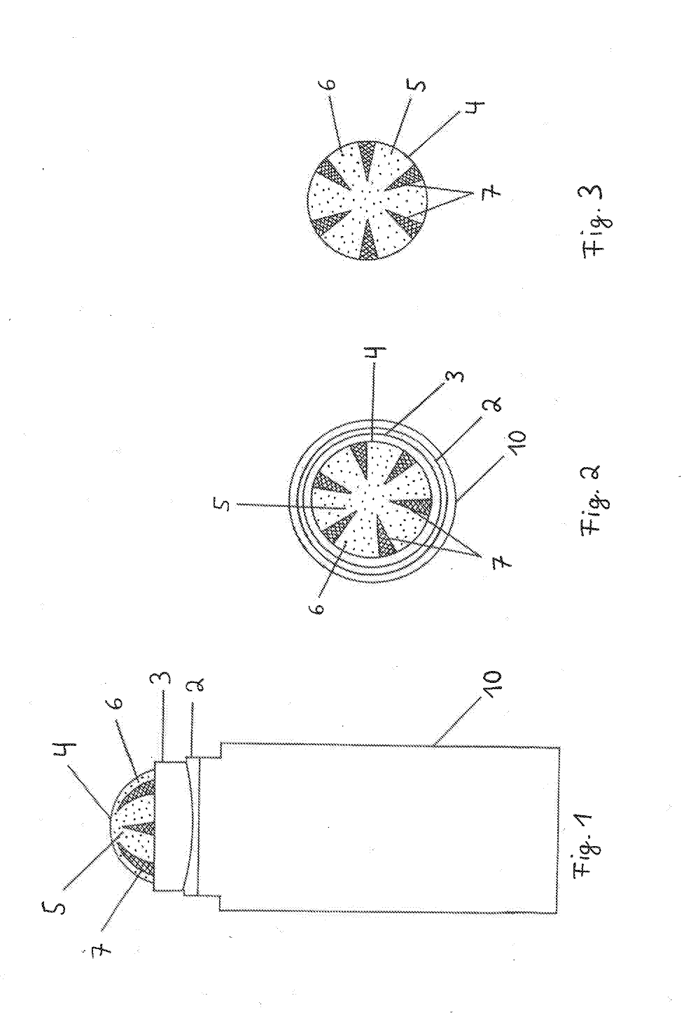

FIG. 1 shows a side view of a roll-on stick 1 with an attachment element 2. The attachment element 2 is intended for applying substances, for example viscous liquids, onto the skin and consists of two receiving shells 3 and a body 4 freely moving in the receiving shells 3. The attachment element 2 is provided for screwing it onto a container 10 where the substance to be applied is located. In the shown embodiment, the movable body 4 consists of a free rotating ball of which less than 50% project from the attachment element 2 to the top. In the lower region, the ball is nearly completely enclosed by the attachment element 2 so that it may neither fall out to the top nor to the bottom. The ball is in contact with the substance located inside the container 10 and adhering to the ball's surface, and which is, due to the rolling motion, transported to the outside and may be transferred, for example, onto the skin.

The receiving shells 3 are here an integral part of the attachment element 2 and may be integrally equipped with the required internal thread, or after the joining of the two receiving shells 3, the required thread collar may be optionally formed. As an alternative, it is of course possible that instead of a thread, a different connection with the container 10 is employed. For example, the lower region of the receiving shells 3 may be glued or welded to the container 10, in particular by laser welding. To insert the rotationally symmetric body 4, in particular the shown ball, the two receiving shells 3 may be pressed, glued or also welded after the ball has been inserted. Instead of a ball, it would also be conceivable that a cylindrical roller or ellipse is used for the roll-on stick 1.

Corresponding to the present invention, the spherical-symmetrical body 4 may be designed in one piece, or for example at least in two pieces. If the body 4 is designed to be hollow, the cavity may be filled with an at least partially transparent liquid by well-known techniques. These may be liquids which are coloured and do not intermingle, or, if they are intermingled, assume their original state after a certain time. It is furthermore possible to fill the cavity of the ball with solid particles 6, for example in the form of snow flakes which are swirled around due to the rotation of the ball and thus cause the effect, for example, of a snow globe. Instead of flakes, in particular twinkling flakes or luminescent flakes, the particles may be employed in any shape serving a decorative purpose or used, for example, as an advertising medium.

As soon as the roll-on stick 1 is used and thereby the body 4 is caused to rotate, the solid particles and the present liquids are swirled so that an attractive effect clearly visible for anyone is formed which may be very well utilized as an advertising medium.

Moreover, the existing cavity in the body may be divided by several partitions 7 so that different-coloured liquids or else different-coloured decorative elements may be employed. It is also possible that one part of the spaces is filled with liquids, while another part is filled with solid particles.

The roll-on stick 1 may thus be employed for decorative or commercial purposes, for example as advertising article or for reproducing a certain brand name, optionally a flag, or it may contain a writing indicating a particular event. The solid particles to be introduced may have any desired structure. For example, it is also possible to create an image engraved by laser within the ball which will rotate when in use. It is also possible to use a certain pattern, for example a net structure or the like, which also leads to an impressive viewing object due to the rotation.

To improve the transport of the substance, the body 4 may be provided with a rough surface or indentations in which the substance will gather and may thus be transferred to the skin. Here, it is easily possible that a three-dimensional or any desired structure is engraved also on the surface of the body 4 which improves, on the one hand, the advertising effect, and on the other hand the transport of the substances.

FIG. 2 shows the roll-on stick 1 in a plan view where one can only see the outer edge of the container 10, and the body 4 and the support consisting of the two receiving shells 3 are centrically screwed onto it.

FIG. 3 shows, in a single view, the body 4 in the form of a ball of which the surface is subdivided with partitions 7 so that different liquids may be embedded in the ball if the partitions are closed partitions 7, or the partitions lead to an amplification of the swirling.

### LIST OF REFERENCE NUMERALS

1 roll-on stick
2 attachment element
3 receiving shell
4 body
5 cavity
6 solid particles
7 partition
10 container

1. Attachment element (2) for a container (10) for applying a substance, in particular liquids, free-flowing formulations, pastes or powders, comprising at least one rotationally symmetric body (4) free moving in a support for applying the substance in particular to a skin surface,
characterized in that
the body (4) comprises a sealable cavity (5) and consists of at least partially transparent material, wherein the body (4) is at least partially filled with an at least partially transparent liquid and/or solid particles (6).

2. Attachment element (2) according to claim 1,
characterized in that
the body (4) is designed as a spherical, cylindrical or ellipsoid body.

3. Attachment element (2) according to claim 1,
characterized in that
the cavity (5) is subdivided by at least one partition (7).

4. Attachment element (2) according to claim 1,
characterized in that
the solid particles (6) consist of small, freely moving particles, for example mica, pearls, stars and coloured flakes.

5. Attachment element (2) according to claim 1,
characterized in that
the solid particles (6) consist of small figurines or structures.

6. Attachment element (2) according to claim 1,
characterized in that
the body (4) consists of glass or plastics.

7. Attachment element (2) according to claim 1,
characterized in that
the body (4) at least partially has a rough surface.

8. Attachment element (2) according to claim 7,
characterized in that
the surface is designed to be rough by a structuring.

9. Attachment element (2) according to claim 8,
characterized in that
the structuring on the surface of the body (4) consists of engraved motives, marks, brand names or of indentations and elevations.

10. Attachment element (2) according to claim 1,
characterized in that
the surface of the body (4) is at least partially coloured.
